(19) United States
(12) Patent Application Publication
(10) Pub. No.: US 2025/0221624 A1
(43) Pub. Date: Jul. 10, 2025

(54) IMAGE DIAGNOSTIC SYSTEM, IMAGE DIAGNOSTIC METHOD, AND STORAGE MEDIUM

(71) Applicant: TERUMO KABUSHIKI KAISHA, Tokyo (JP)

(72) Inventor: Yuki SAKAGUCHI, Fujisawa Kanagawa (JP)

(21) Appl. No.: 19/094,743

(22) Filed: Mar. 28, 2025

Related U.S. Application Data
(63) Continuation of application No. PCT/JP2023/035281, filed on Sep. 27, 2023.

(30) Foreign Application Priority Data
Sep. 29, 2022 (JP) 2022-156624

(51) Int. Cl. A61B 5/00; G06T 7/11; G06T 7/13; G06T 7/62

(52) U.S. Cl. CPC A61B 5/0073; A61B 5/0084; A61B 5/6852; A61B 5/742; G06T 7/11; G06T 7/13; G06T 7/62; A61B 2560/0462; A61B 2576/02; G06T 2207/10072; G06T 2207/20081; G06T 2207/20172; G06T 2207/30101

(57) ABSTRACT

An image diagnostic system includes a catheter insertable into a blood vessel, a memory that stores a program, a display, and a processor configured to execute the program to: control the catheter to acquire a tomographic image of a blood vessel, determine a first region corresponding to a main trunk of the blood vessel and a second region corresponding to a side branch of the blood vessel in the acquired image, and determine whether the first and second regions are connected to each other, upon determining that the first and second regions are connected to each other, determine a first contour line of the first region in the acquired image, determine a second contour line of the second region in the acquired image based on the first contour line, and control the display to display the first and second contour lines on the acquired image using different display modes.

### CROSS-REFERENCE TO RELATED APPLICATION

This application is a continuation of International Patent Application No. PCT/JP2023/035281 filed Sep. 27, 2023, which is based upon and claims the benefit of priority from Japanese Patent Application No. 2022-156624, filed Sep. 29, 2022, the entire contents of which are incorporated herein by reference.

### BACKGROUND

### Technical Field

Embodiments described herein relate to an image diagnostic system, an image diagnostic method, and a storage medium.

### Related Art

A tomographic image including an ultrasonic tomographic image of a blood vessel is generated by an intravascular ultrasound (IVUS) method using a catheter, and an ultrasonic inspection of the blood vessel is performed based on the generated tomographic image. For the purpose of assisting a doctor to make a diagnosis, a technology of adding information to such a tomographic image by image processing or machine learning has been developed. In the conventional technique, features of a blood vessel such as a luminal wall and a stent can be individually extracted from the blood vessel image.

When a blood vessel is observed using a tomographic image, a main trunk and a side branch branching from the main trunk may appear. Since the long axis of the catheter placed in the main trunk is not parallel to the long axis of the side branch, the side branch shape is drawn in various forms depending on the observation position. In particular, it is necessary to virtually define the region of the branch portion where the main trunk and the side branch are connected, but doing so is challenging.

### SUMMARY

In one aspect, there are provided an image diagnostic system, an image diagnostic method, and a storage medium capable of automatically visualizing a branch portion of a blood vessel where a main trunk and a side branch are connected to each other.

In one embodiment, an image diagnostic system comprises: a catheter insertable into a blood vessel; a memory that stores a program; a display; and a processor configured to execute the program to: control the catheter to acquire a tomographic image of a blood vessel, determine a first region corresponding to a main trunk of the blood vessel and a second region corresponding to a side branch of the blood vessel in the acquired image, and determine whether the first and second regions are connected to each other, upon determining that the first and second regions are connected to each other, determine a first contour line of the first region in the acquired image, determine a second contour line of the second region in the acquired image based on the first contour line, and control the display to display the first and second contour lines on the acquired image using different display modes.

In one aspect, it is possible to automatically visualizing a branch portion of a blood vessel where a main trunk and a side branch are connected to each other.

### BRIEF DESCRIPTION OF DRAWINGS

FIG. 1 is a schematic diagram illustrating a configuration of an image diagnosis system according to a first embodiment.

FIG. 2 is a schematic diagram illustrating an image diagnosis catheter.

FIG. 3 is a diagram illustrating a cross section of a blood vessel through which a sensor unit is inserted.

FIG. 4A is a diagram for explaining a tomographic image.

FIG. 4B is a diagram for explaining a tomographic image.

FIG. 5 is a schematic diagram illustrating a side branch shape.

FIG. 6 is a block diagram illustrating a configuration of an image processing apparatus.

FIG. 7 is a schematic diagram illustrating a configuration of a learning model

FIG. 8 is a flowchart for explaining a procedure executed by the image processing apparatus.

FIG. 9A is a schematic diagram illustrating a display example of a contour line.

FIG. 9B is a schematic diagram illustrating a display example of a contour line.

FIG. 9C is a schematic diagram illustrating a display example of a contour line.

FIG. 10 is a flowchart illustrating a procedure for determining the contour line of a side branch region with reference to the contour line of a main trunk region.

FIG. 11 is a diagram for explaining a boundary portion correction method.

FIG. 12 is a flowchart illustrating a determination procedure of a contour line in a case where a side branch comes into contact with a visual field boundary.

FIG. 13 is a flowchart illustrating a procedure for determining a contour line of a side branch region with reference to a boundary line of a visual field boundary.

FIG. 14 is a schematic diagram illustrating selection of contour points in a side branch region.

FIG. 15 is a schematic diagram illustrating a contour line outside a visual field region by a virtual line.

FIG. 16 is a diagram for explaining a work environment of annotation.

### DETAILED DESCRIPTION

Hereinafter, embodiments of the present invention will be described in detail with reference to the drawings.

### First Embodiment

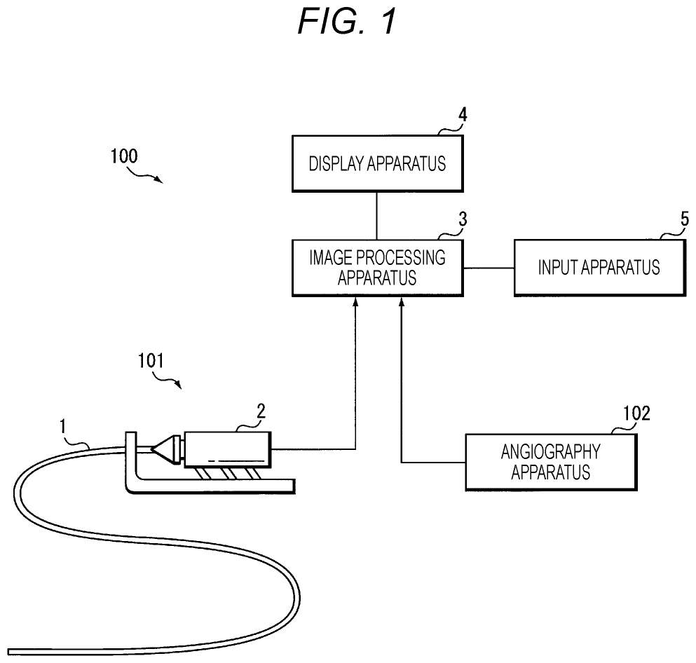

FIG. 1 is a schematic diagram illustrating a configuration of an image diagnosis system 100 according to a first embodiment. In the present embodiment, the image diagnosis system 100 with a dual type catheter having functions of both intravascular ultrasound diagnosis method (IVUS) and optical coherence tomography (OCT) will be described. In the dual type catheter, a mode of acquiring an ultrasonic tomographic image only by IVUS, a mode of acquiring an optical coherence tomographic image only by OCT, and a mode of acquiring both tomographic images by IVUS and OCT are provided, and these modes can be switched and used. Hereinafter, the ultrasonic tomographic image and the optical coherence tomographic image are also referred to as an IVUS image and an OCT image, respectively. The IVUS image and the OCT image are examples of tomographic images of a blood vessel, and in a case where it is not necessary to distinguish and describe the IVUS image and the OCT image, they are also simply described as tomographic images.

The image diagnosis system 100 includes an intravascular inspection apparatus 101, an angiography apparatus 102, an image processing apparatus 3, a display apparatus 4, and an input apparatus 5. The intravascular inspection apparatus 101 includes an image diagnosis catheter 1 and a motor drive unit (MDU) 2. The image diagnosis catheter 1 is connected to the image processing apparatus 3 via the MDU 2. The display apparatus 4 and the input apparatus 5 are connected to the image processing apparatus 3. The display apparatus 4 is, for example, a liquid crystal display, an organic EL display, or the like, and the input apparatus 5 is, for example, a keyboard, a mouse, a touch panel, a microphone, or the like. The input apparatus 5 and the image processing apparatus 3 may be integrated into a single apparatus. Furthermore, the input apparatus 5 may be a sensor that receives a gesture input, a line-of-sight input, or the like.

The angiography apparatus 102 is connected to the image processing apparatus 3. The angiography apparatus 102 images a blood vessel from outside a living body of a patient using X-rays while injecting a contrast agent into the blood vessel of the patient to obtain an angiographic image that is a fluoroscopic image of the blood vessel. The angiography apparatus 102 includes an X-ray source and an X-ray sensor, and images an X-ray fluoroscopic image of the patient by the X-ray sensor receiving X-rays emitted from the X-ray source. Note that the image diagnosis catheter 1 has a marker that does not transmit X-rays, and the position of the image diagnosis catheter 1 (i.e., the marker) is visualized in the angiographic image. The angiography apparatus 102 outputs the angiographic image obtained by imaging to the image processing apparatus 3, and causes the display apparatus 4 to display the angiographic image via the image processing apparatus 3. Note that the display apparatus 4 displays the angiographic image and the tomographic image imaged using the image diagnosis catheter 1.

Note that, in the present embodiment, the image processing apparatus 3 is connected to the angiography apparatus 102 that images two-dimensional angiographic images. However, the present invention is not limited to the angiography apparatus 102 as long as it is an apparatus that images a luminal organ of a patient and the image diagnosis catheter 1 from a plurality of directions outside the living body.

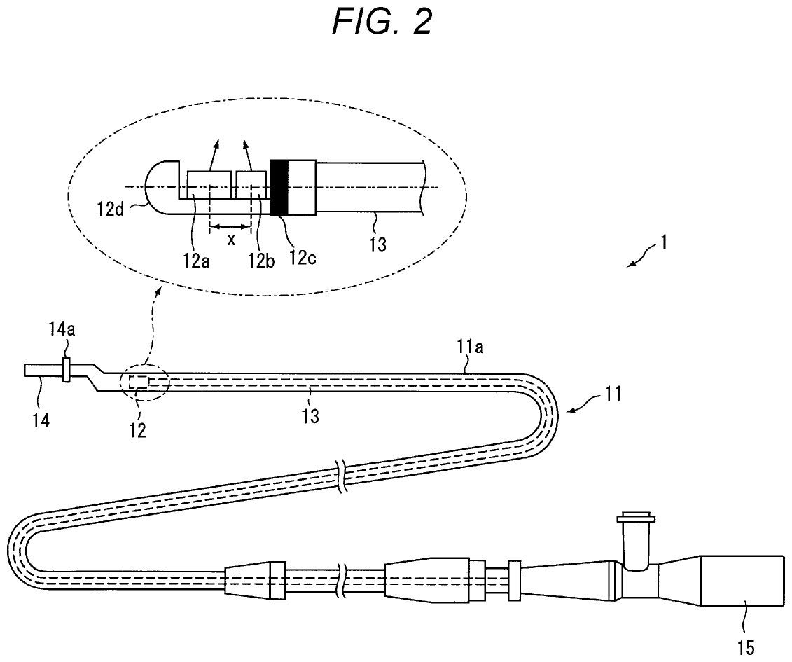

FIG. 2 is a schematic diagram illustrating the image diagnosis catheter 1. Note that a region indicated by a one-dot chain line on an upper side in FIG. 2 is an enlarged view of a region indicated by a one-dot chain line on a lower side. The image diagnosis catheter 1 includes a probe 11 and a connector portion 15 disposed at an end of the probe 11. The probe 11 is connected to the MDU 2 via the connector portion 15. In the following description, a side far from the connector portion 15 of the image diagnosis catheter 1 will be referred to as a distal end side, and a side of the connector portion 15 will be referred to as a proximal end side. The probe 11 includes a catheter sheath 11a, and a guide wire insertion portion 14 through which a guide wire can be inserted is provided at a distal portion thereof. The guide wire insertion portion 14 forms a guide wire lumen, receives a guide wire previously inserted into a blood vessel, and guides the probe 11 to an affected part by the guide wire. The catheter sheath 11a forms a tube portion continuous from a connection portion with the guide wire insertion portion 14 to a connection portion with the connector portion 15. A shaft 13 is inserted into the catheter sheath 11a, and a sensor unit 12 is connected to a distal end side of the shaft 13.

The sensor unit 12 includes a housing 12d, and a distal end side of the housing 12d is formed in a hemispherical shape in order to suppress friction and catching with an inner surface of the catheter sheath 11a. In the housing 12d, an ultrasound transmitter and receiver 12a (hereinafter referred to as an IVUS sensor 12a) that transmits ultrasonic waves into a blood vessel and receives reflected waves from the blood vessel and an optical transmitter and receiver 12b (hereinafter referred to as an OCT sensor 12b) that transmits near-infrared light into the blood vessel and receives reflected light from the inside of the blood vessel are disposed. In the example illustrated in FIG. 2, the IVUS sensor 12a is provided on the distal end side of the probe 11, the OCT sensor 12b is provided on the proximal end side thereof, and the IVUS sensor 12a and the OCT sensor 12b are arranged apart from each other by a distance x along the axial direction on a central axis (on a two-dot chain line in FIG. 2) of the shaft 13. In the image diagnosis catheter 1, the IVUS sensor 12a and the OCT sensor 12b are attached such that a direction that is approximately 90 degrees with respect to the axial direction of the shaft 13 (i.e., the radial direction of the shaft 13) is set as a transmission/reception direction of an ultrasonic wave or near-infrared light. Note that the IVUS sensor 12a and the OCT sensor 12b are desirably attached slightly shifted from the radial direction so as not to receive a reflected wave or reflected light on the inner surface of the catheter sheath 11a. In the present embodiment, for example, as indicated by an arrow in FIG. 2, the IVUS sensor 12a is attached with a direction inclined to the proximal end side with respect to a radial direction as an irradiation direction of the ultrasonic wave, and the OCT sensor 12b is attached with a direction inclined to the distal end side with respect to the radial direction as an irradiation direction of the near-infrared light.

An electric signal cable (not illustrated) connected to the IVUS sensor 12a and an optical fiber cable (not illustrated) connected to the OCT sensor 12b are inserted into the shaft 13. The probe 11 is inserted into the blood vessel from the distal end side. The sensor unit 12 and the shaft 13 can move forward or rearward inside the catheter sheath 11a and can rotate in a circumferential direction. The sensor unit 12 and the shaft 13 rotate about the central axis of the shaft 13 as a rotation axis. In the image diagnosis system 100, by using an imaging core including the sensor unit 12 and the shaft 13, a state inside the blood vessel is measured by an ultrasonic tomographic image (IVUS image) captured from the inside of the blood vessel or an optical coherence tomographic image (i.e., an OCT image) captured from the inside of the blood vessel.

The MDU 2 is a drive apparatus to which the probe 11 is detachably attached by the connector portion 15, and controls the operation of the image diagnosis catheter 1 inserted into the blood vessel by driving a built-in motor according to an operation of a medical worker. For example, the MDU 2 performs a pull-back operation of rotating the sensor unit 12 and the shaft 13 inserted into the probe 11 in the circumferential direction while pulling the sensor unit 12 and the shaft 13 toward the MDU 2 side at a constant speed. The sensor unit 12 continuously scans the inside of the blood vessel at predetermined time intervals while moving and rotating from the distal end side to the proximal end side by the pull-back operation and continuously captures a plurality of transverse tomographic images substantially perpendicular to the probe 11 at predetermined intervals. The MDU 2 outputs reflected wave data of an ultrasonic wave received by the IVUS sensor 12a and reflected light data received by the OCT sensor 12b to the image processing apparatus 3.

The image processing apparatus 3 acquires a signal data set which is the reflected wave data of the ultrasonic wave received by the IVUS sensor 12a and a signal data set which is reflected light data received by the OCT sensor 12b via the MDU 2. The image processing apparatus 3 generates ultrasonic line data from a signal data set of the ultrasonic waves, and constructs an ultrasonic tomographic image (i.e., an IVUS image) obtained by imaging a transverse section of the blood vessel based on the generated ultrasonic line data. In addition, the image processing apparatus 3 generates optical line data from the signal data set of the reflected light, and constructs an optical coherence tomographic image (i.e., an OCT image) obtained by imaging a transverse section of the blood vessel based on the generated optical line data. Here, the signal data set acquired by the IVUS sensor 12a and the OCT sensor 12b and the tomographic image constructed from the signal data set will be described.

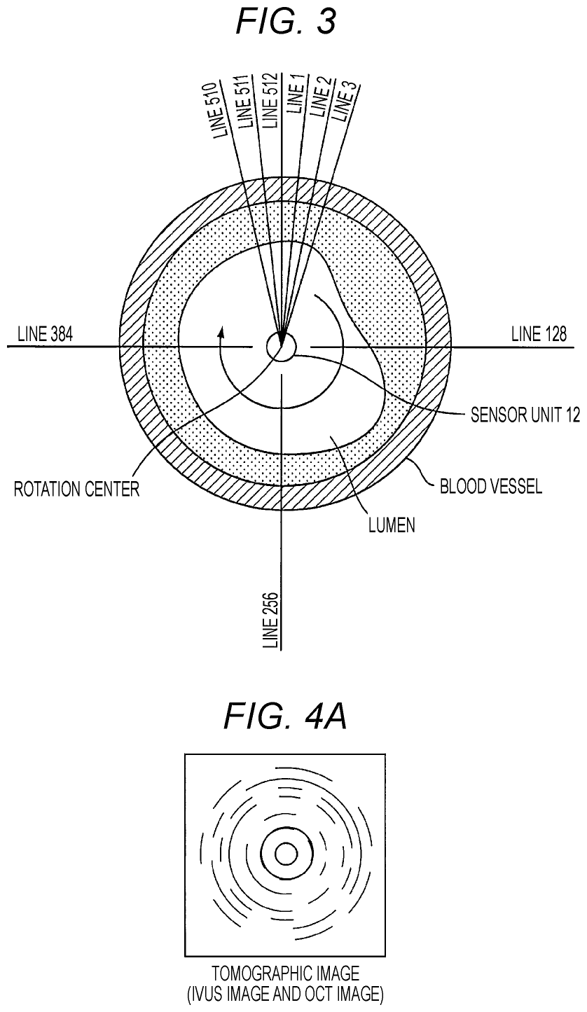

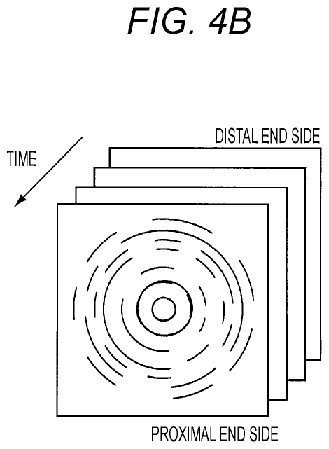

FIG. 3 is a diagram for explaining a cross section of a blood vessel through which the sensor unit 12 is inserted, and FIGS. 4A and 4B are diagrams for explaining tomographic images. First, with reference to FIG. 3, operations of the IVUS sensor 12a and the OCT sensor 12b in the blood vessel, and signal data sets (e.g., ultrasonic line data and optical line data) acquired by the IVUS sensor 12a and the OCT sensor 12b will be described. When the imaging of the tomographic image is started in a state where the imaging core is inserted into the blood vessel, the imaging core rotates about a central axis of the shaft 13 as a rotation center in a direction indicated by an arrow. At this time, the IVUS sensor 12a transmits and receives an ultrasonic wave at each rotation angle. Lines 1, 2, . . . 512 indicate transmission/reception directions of ultrasonic waves at each rotation angle. In the present embodiment, the IVUS sensor 12a intermittently transmits and receives ultrasonic waves 512 times while rotating 360 degrees (i.e., 1 rotation) in the blood vessel. Since the IVUS sensor 12a acquires data of one line in the transmission/reception direction by transmitting and receiving an ultrasonic wave once, it is possible to obtain 512 pieces of ultrasonic line data radially extending from the rotation center during one rotation. The 512 pieces of ultrasonic line data are dense in the vicinity of the rotation center, but become sparse with distance from the rotation center. Therefore, the image processing apparatus 3 can generate a two-dimensional ultrasonic tomographic image (i.e., an IVUS image) as illustrated in FIG. 4A by generating pixels in an empty space of each line by known interpolation processing.

Similarly, the OCT sensor 12b also transmits and receives the measurement light at each rotation angle. Since the OCT sensor 12b also transmits and receives the measurement light 512 times while rotating 360 degrees in the blood vessel, it is possible to obtain 512 pieces of optical line data radially extending from the rotation center during one rotation. Moreover, for the optical line data, the image processing apparatus 3 can generate a two-dimensional optical coherence tomographic image (i.e., an OCT image) similar to the IVUS image illustrated in FIG. 4A by generating pixels in an empty space of each line by known interpolation processing. That is, the image processing apparatus 3 generates optical line data based on interference light generated by causing reflected light and, for example, reference light obtained by separating light from a light source in the image processing apparatus 3 to interfere with each other, and constructs an optical coherence tomographic image obtained by imaging the transverse section of the blood vessel based on the generated optical line data.

The two-dimensional tomographic image generated from the 512 pieces of line data in this manner is referred to as an IVUS image or an OCT image of one frame. Note that, since the sensor unit 12 scans while moving in the blood vessel, an IVUS image or an OCT image of one frame is acquired at each position rotated once within a movement range. That is, since the IVUS image or the OCT image of one frame is acquired at each position from the distal end side to the proximal end side of the probe 11 in the movement range, as illustrated in FIG. 4B, the IVUS image or the OCT image of a plurality of frames is acquired within the movement range.

The image diagnosis catheter 1 has a marker that does not transmit X-rays in order to confirm a positional relationship between the IVUS image obtained by the IVUS sensor 12a or the OCT image obtained by the OCT sensor 12b and the angiographic image obtained by the angiography apparatus 102. In the example illustrated in FIG. 2, a marker 14a is provided at the distal portion of the catheter sheath 11a, for example, the guide wire insertion portion 14, and a marker 12c is provided on the shaft 13 side of the sensor unit 12. When the image diagnosis catheter 1 configured as described above is imaged with X-rays, an angiographic image in which the markers 14a and 12c are visualized is obtained. The positions of the markers 14a and 12c are mere examples, and the marker 12c may be provided on the shaft 13 instead of the sensor unit 12, and the marker 14a may be provided at a portion other than the distal portion of the catheter sheath 11a.

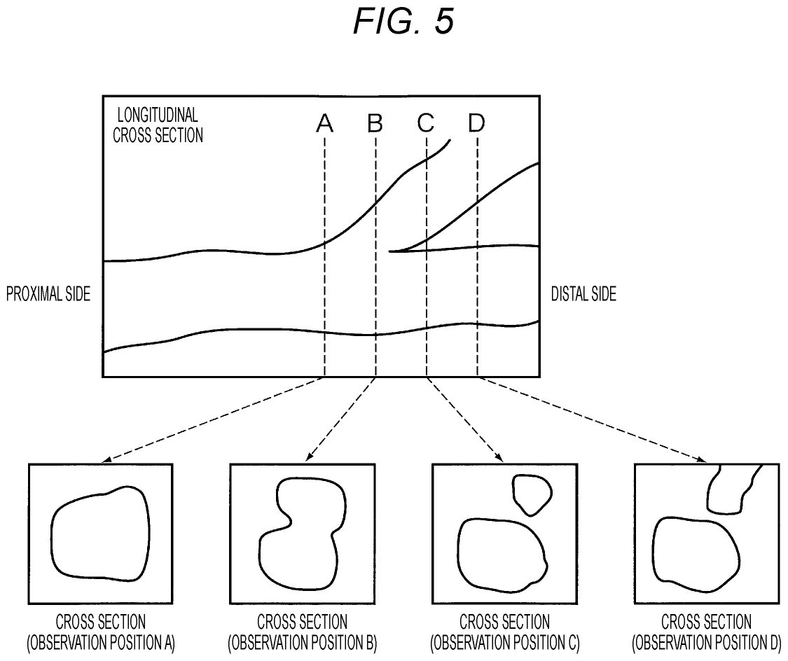

FIG. 5 is a schematic diagram illustrating a side branch shape. In the tomographic image obtained by the intravascular inspection apparatus 101, the tomographic structure of the blood vessel into which the image diagnosis catheter 1 is inserted appears. Hereinafter, the blood vessel into which the image diagnosis catheter 1 is inserted is referred to as a main trunk. In addition, a tomographic structure of a blood vessel branching from the main trunk may appear in the tomographic image. Hereinafter, a blood vessel branching and extending from the main trunk is referred to as a side branch. Since the long axis of the image diagnosis catheter 1 inserted into the main trunk and the long axis of the side branch are not generally parallel, the side branch shape is drawn in various forms depending on the observation position.

The longitudinal cross-sectional view illustrated in FIG. 5 illustrates an example of a blood vessel having a main trunk and a side branch branching and extending from the main trunk. When the cross section of the blood vessel is observed at the observation position A, only the main trunk is drawn and side branches do not appear. On the other hand, when observed at the observation position B, the main trunk and the side branch are drawn in a connected state. In addition, when observed at the observation position C, the main trunk and the side branch are drawn as independent regions. Further, when observed at the observation position D, the main trunk and the side branch are drawn as independent regions, and the side branch extends to the outside of the visual field region.

When acquiring a tomographic image of a blood vessel, the image processing apparatus 3 according to the present embodiment extracts or determines a main trunk region from the acquired tomographic image. In addition, when the acquired tomographic image includes a side branch region in addition to the main trunk region, the image processing apparatus 3 extracts or determines both the main trunk region and the side branch region. In a case where the main trunk region and the side branch region are connected, the image processing apparatus 3 defines boundary lines thereof and presents the boundary lines to the user as individual regions.

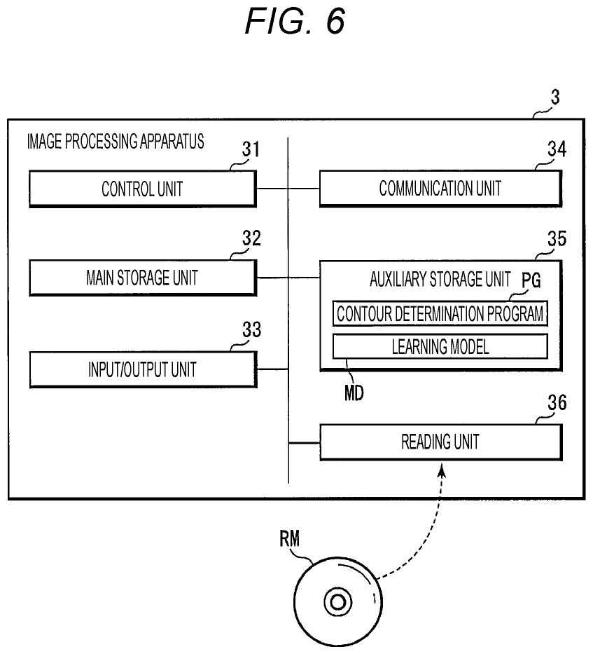

FIG. 6 is a block diagram illustrating a configuration of the image processing apparatus 3. The image processing apparatus 3 includes a control unit 31, a main storage unit 32, an input/output unit 33, a communication unit 34, an auxiliary storage unit 35, and a reading unit 36. The image processing apparatus 3 is not limited to a single apparatus, and may be formed by a plurality of apparatus. In addition, the image processing apparatus 3 may be a server client system, a cloud server, or virtual machine virtually constructed by software. In the following description, it is assumed that the image processing apparatus 3 is a single apparatus.

The control unit 31 includes one or more processors such as central processing units (CPU), micro processing units (MPU), graphics processing units (GPU), general purpose computing on graphics processing units (GPGPU), and tensor processing units (TPU). The control unit 31 is connected to each hardware unit constituting the image processing apparatus 3 via a bus.

The main storage unit 32, which is a temporary memory area such as a static random access memory (SRAM), a dynamic random access memory (DRAM), or a flash memory, temporarily stores data necessary for the control unit 31 to execute arithmetic processing.

The input/output unit 33 includes an interface circuit that connects external apparatuses such as the intravascular inspection apparatus 101, the angiography apparatus 102, the display apparatus 4, and the input apparatus 5. The control unit 31 acquires an IVUS image and an OCT image from the intravascular inspection apparatus 101 via the input/output unit 33, and acquires an angiographic image from the angiography apparatus 102. In addition, the control unit 31 outputs a medical image signal of an IVUS image, an OCT image, or an angiographic image to the display apparatus 4 via the input/output unit 33, thereby displaying the medical image on the display apparatus 4. Furthermore, the control unit 31 receives information input to the input apparatus 5 via the input/output unit 33.

The communication unit 34 includes, for example, a communication interface circuit conforming to a communication standard such as 4G, 5G, or WiFi. The image processing apparatus 3 communicates with an external server such as a cloud server connected to an external network such as the Internet via the communication unit 34. The control unit 31 may access an external server via the communication unit 34 and refer to various data stored in a storage of the external server. Furthermore, the control unit 31 may cooperatively perform the processing in the present embodiment by performing, for example, inter-process communication with the external server.

The auxiliary storage unit 35 is a storage device such as a hard disk drive (HDD) or a solid state drive (SSD). The auxiliary storage unit 35 stores a computer program executed by the control unit 31 and various data necessary for processing of the control unit 31. Note that the auxiliary storage unit 35 may be an external storage device connected to the image processing apparatus 3. The computer program executed by the control unit 31 may be written in the auxiliary storage unit 35 at the manufacturing stage of the image processing apparatus 3, or the computer program distributed by a remote server apparatus may be acquired by the image processing apparatus 3 through communication and stored in the auxiliary storage unit 35. The computer program may be readably recorded in a recording medium RM such as a magnetic disk, an optical disk, or a semiconductor memory, or may be read from the recording medium RM by the reading unit 36 and stored in the auxiliary storage unit 35. An example of the computer program stored in the auxiliary storage unit 35 is a contour determination program PG for causing a computer to execute processing of determining contour lines of the main trunk region and the side branch region from a tomographic image of a blood vessel and displaying the determined contour lines.

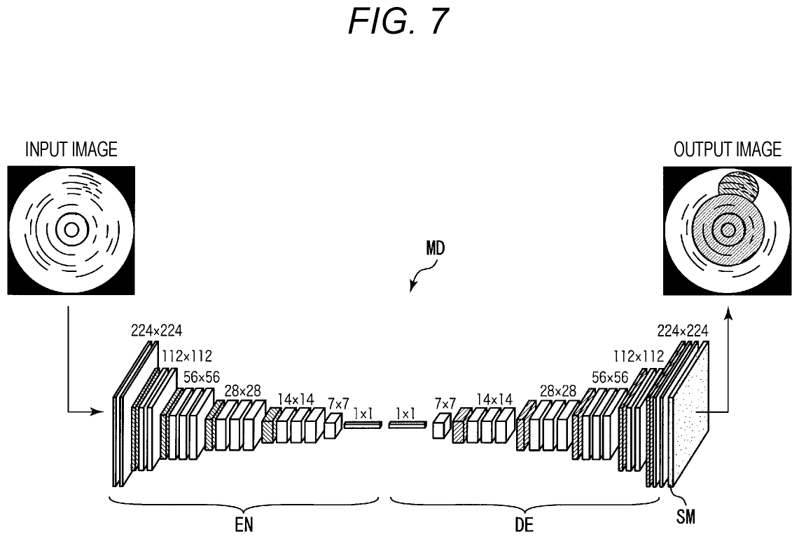

In addition, the auxiliary storage unit 35 stores a learning model MD used for processing of recognizing the main trunk region and the side branch region from the tomographic image of the blood vessel. FIG. 7 is a schematic diagram illustrating a configuration of the learning model MD. The learning model MD is a computer learning model for performing image segmentation, and is constructed by, for example, a neural network including a convolution layer such as SegNet. Alternatively, the learning model MD is not limited to SegNet, and may be a learning model for image segmentation such as a fully convolutional network (FCN), a U-shaped network (U-Net), or a Pyramid Scene Parsing Network (PSPNet). Furthermore, the learning model MD may be a learning model for object detection such as You Only Look Once (YOLO), a Single Shot Multi-Box Detector (SSD), or a Vision Transformer (ViT).

In the present embodiment, the input image to the learning model MD is a tomographic image (i.e., an IVUS image or an OCT image) of a blood vessel obtained by the intravascular inspection apparatus 101. The learning model MD is learned so as to output information indicating a recognition result of the main trunk region and the side branch region included in the tomographic image in response to the input of the tomographic image. The learning model MD is described by its definition information. The definition information includes information on layers constituting the learning model MD, information on nodes constituting each layer, and internal parameters such as a weight coefficient and bias between nodes. The internal parameters are learned by a predetermined learning algorithm. The auxiliary storage unit 35 stores definition information of the learning model MD including trained internal parameters.

The learning model MD in the present embodiment includes, for example, an encoder EN, a decoder DE, and a softmax layer SM. The encoder EN is configured by alternately arranging a convolution layer and a pooling layer. The convolution layer is multilayered into two to three layers.

In the convolution layer, a convolution operation of input data and a filter having a size (for example, 3×3, 5×5, or the like) determined in each of the input data and the filter is performed. That is, an input value input to a position corresponding to each element of the filter and a weight coefficient set in advance for the filter are multiplied for each element, and a linear sum of multiplication values for each element is calculated. An output in the convolution layer is obtained by adding the set bias to the calculated linear sum. Note that the result of the convolution operation may be converted by an activation function. For example, a rectified linear unit (ReLU) can be used as the activation function. The output of the convolution layer represents a feature map from which the features of the input data are extracted.

In the pooling layer, local statistical values of the feature map output from the convolution layer which is an upper layer connected to the input side are calculated. Specifically, a window having a predetermined size (for example, 2×2, 3×3) corresponding to the position of the upper layer is set, and the local statistical values are calculated from the input value in the window. As the statistical values, for example, a maximum value can be adopted. The size of the feature map output from the pooling layer is reduced (i.e., downsampled) according to the size of the window. The example of FIG. 7 illustrates that the input image of 224 pixels×224 pixels is sequentially downsampled into 112×112, 56×56, 28×28, . . . , and 1×1 feature maps by sequentially repeating the computation in the convolution layer and the computation in the pooling layer in the encoder 310.

The output of the encoder EN is input to the decoder DE. The decoder DE is configured by alternately arranging a deconvolution layer and an unpooling layer. The deconvolution layer is multilayered into two to three layers.

In the deconvolution layer, a deconvolution operation is performed on the input feature map. The deconvolution operation is computation of restoring a feature map before a convolution operation is performed under estimation that an input feature map is a result of the convolution operation using a specific filter. In this computation, when a specific filter is represented by a matrix, a product of a transposed matrix for the matrix and the input feature map is calculated to generate a feature map for output. Note that the computation result of the deconvolution layer may be converted by the activation function such as ReLU described above.

The unpooling layers included in the decoder DE are individually associated with the pooling layers included in the encoder EN on a one-to-one basis, and the associated pairs have substantially the same size. The unpooling layer again enlarges (i.e., upsamples) the size of the downsampled feature map in the pooling layer of the encoder EN. The example of FIG. 7 illustrates that upsampling is sequentially performed on the feature maps of 1×1, 7×7, 14×14, . . . , and 224×224 by sequentially repeating the computation in the convolution layer and the computation in the pooling layer in the decoder 320.

The output of the decoder DE is input to the softmax layer SM. The softmax layer SM applies the softmax function to the input value from the deconvolution layer connected to the input side, thereby outputting the probability of the label for identifying the site at each position or pixel. In the present embodiment, labels for identifying the main trunk and the side branches are set. The control unit 31 of the image processing apparatus 3 refers to the probability of the label output from the softmax layer SM, and recognizes the region belonging to the main trunk (i.e., the main trunk region) and the region belonging to the side branch (i.e., the side branch region) from the tomographic image. The control unit 31 may recognize a region that does not belong to the main trunk region or the side branch region as the background region.

Hereinafter, the operation of the image processing apparatus 3 will be described.

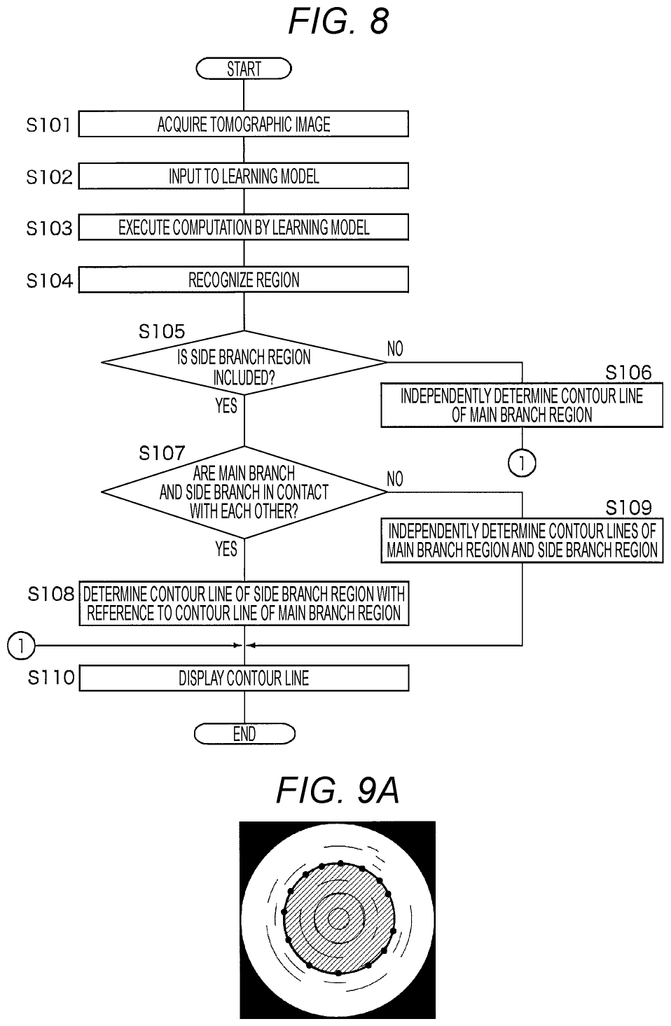

FIG. 8 is a flowchart for explaining a procedure executed by the image processing apparatus 3. The control unit 31 of the image processing apparatus 3 executes the following processing each time a tomographic image is acquired from the intravascular inspection apparatus 101. When acquiring the tomographic image of the blood vessel through the input/output unit 33 (S101), the control unit 31 inputs the acquired tomographic image to the learning model MD (S102), and executes computation using the learning model MD (S103).

The control unit 31 recognizes a region based on a computation result by the learning model MD (S104). Specifically, the control unit 31 determines whether each pixel belongs to a main trunk or a side branch based on the probability of the label for each pixel output from the softmax layer SM of the learning model MD. The control unit 31 recognizes a set of pixels determined to belong to a main trunk as a main trunk region, and recognizes a set of pixels determined to belong to a side branch as a side branch region. The control unit 31 recognizes a set of pixels that do not belong to either a main trunk or a side branch as a background region.

The control unit 31 determines whether a side branch region is included in the region recognized in S104 (S105). When it is determined that a side branch region is not included (S105: NO), the contour line of a main trunk region included in the tomographic image is independently determined (S106). For example, the control unit 31 determines the contour line of a main trunk region by selecting a plurality of points on the peripheral edge of the main trunk region and obtaining an approximate curve such as a spline curve passing through the selected plurality of points (or the vicinity of the points).

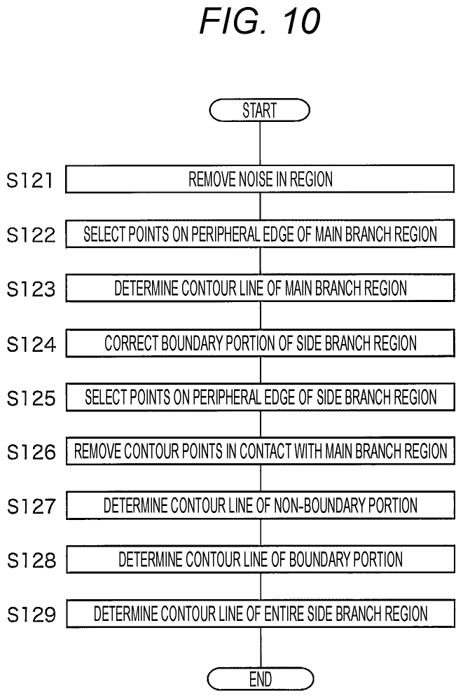

When it is determined that a main trunk region and a side branch region are included (S105: YES), the control unit 31 determines whether the main trunk region and the side branch region are in contact with each other (S107). When it is determined that the main trunk region and the side branch region are in contact with each other (S107: YES), the control unit 31 determines the contour line of the main trunk region included in the tomographic image, and determines the contour line of the side branch region with reference to the determined contour line of the main trunk region (S108). A procedure for determining the contour line of the side branch region with reference to the contour line of the main trunk region will be described in detail with reference to a flowchart illustrated in FIG. 10.

When it is determined that a main trunk region and a side branch region are not in contact with each other (S107: NO), the control unit 31 independently determines contour lines of a main trunk region and a side branch region included in the tomographic image (S109). For example, the control unit 31 determines the contour line of the main trunk region by selecting a plurality of points on the peripheral edge of the main trunk region and obtaining an approximate curve such as a spline curve passing through the selected plurality of points (or the vicinity of the points). In addition, the control unit 31 determines the contour line of the side branch region by selecting a plurality of points on the peripheral edge of the side branch region and obtaining an approximate curve such as a spline curve passing through the selected plurality of points (or the vicinity of the points).

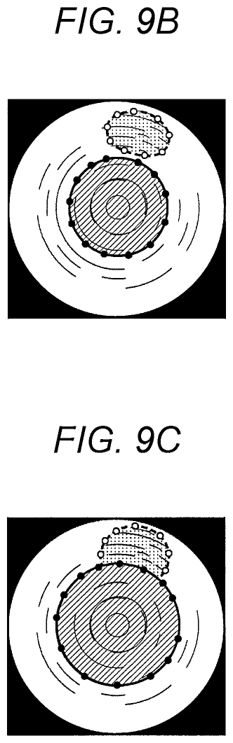

The control unit 31 displays the contour line determined in S106, S108, or S109 (S110). FIGS. 9A to 9C are schematic diagrams illustrating display examples of contour lines. FIG. 9A illustrates an example in which the main trunk region is detected alone. In FIG. 9A, the main trunk region is drawn by a hatched region, and contour points on the peripheral edge of the main trunk region are drawn by black circles. In addition, the contour line of the main trunk region is drawn by a solid line passing through the contour points (or the vicinity of the contour points).

FIG. 9B illustrates an example in which a main trunk region and a side branch region are detected without being in contact with each other. In FIG. 9B, the main trunk region is drawn by a hatched region, and contour points on the peripheral edge of the main trunk region are drawn by black circles. In addition, the contour line of the main trunk region is drawn by a solid line passing through the contour points (or the vicinity of the contour lines). Furthermore, in FIG. 9B, the side branch region is drawn by a dotted region, and contour points on the peripheral edge of the side branch region are drawn by white circles. In addition, the contour line of the side branch region is drawn by a broken line passing through the contour points (or the vicinity of the contour points).

FIG. 9C illustrates an example in which a main trunk region and a side branch region are detected in contact with each other. In FIG. 9C, the main trunk region is drawn by a hatched region, and contour points on the peripheral edge of the main trunk region are drawn by black circles. In addition, the contour line of the main trunk region is drawn by a solid line passing through the contour points (or the vicinity of the contour lines). Furthermore, in FIG. 9C, the side branch region is drawn by a dotted region, and contour points on the peripheral edge of the side branch region are drawn by white circles. In addition, the contour line of the side branch region is drawn by a broken line passing through the contour points (or the vicinity of the contour points). In this example, since the main trunk region and the side branch region are in contact with each other, the contour line of the main trunk region and the contour line of the side branch region are drawn so as to overlap each other at the boundary.

FIGS. 9A to 9C illustrate examples in which the contour line of the main trunk region and the contour line of the side branch region are drawn by the solid line and the broken line, respectively. However, the control unit 31 may change the display mode of the contour line between the main trunk region and the side branch region and display the contour line, for example, by changing the color, or by changing the thickness of the line.

FIG. 10 is a flowchart illustrating a procedure for determining the contour line of a side branch region with reference to the contour line of a main trunk region. The control unit 31 refers to the recognition result of S104 and removes noise in the recognized region (S121). Specifically, in a case where there is an isolated point recognized as a side branch or a background in the region recognized as the main trunk, the control unit 31 performs processing of removing these isolated points. The isolated point may be a single pixel or a small region including a plurality of pixels. The control unit 31 can remove the isolated point by changing the label of the pixel included in the isolated point from “side branch” or “background” to “main trunk”. The same applies to a region recognized as a side branch, and the control unit 31 performs processing of removing an isolated point included in the side branch region.

The control unit 31 selects a plurality of points on the peripheral edge of the main trunk region (S122). In the present embodiment, a set of pixels determined to belong to the main trunk is recognized as the main trunk region. Among the pixels belonging to the main trunk, in a case where all the eight pixels adjacent to the pixel of interest (four on the upper, lower, left, and right sides+four in the oblique direction) belong to the main trunk, the pixel of interest is recognized as a pixel inside the main trunk region, and in a case where one or more of the eight pixels adjacent to the pixel of interest belong to a side branch or a background, the pixel of interest is recognized as a pixel on the peripheral edge. The control unit 31 selects a predetermined number of pixels from among pixels recognized as pixels on the peripheral edge among pixels belonging to the main trunk region. The number of pixels to be selected may be set in advance or may be set according to the size of the main trunk region. The pixel selected in S122 is referred to as a contour point of the main trunk region.

The control unit 31 determines the contour line of the main trunk region based on the plurality of contour points selected in S122 (S123). The control unit 31 can determine the contour line of the main trunk region by obtaining an approximate curve such as a spline curve passing through the contour points (or the vicinity of the contour points) of the main trunk region.

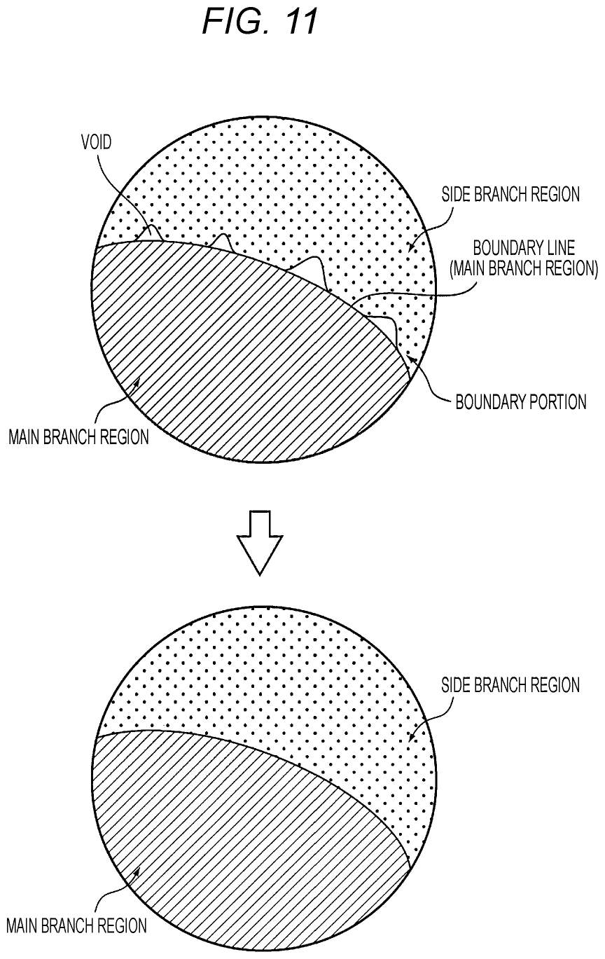

The control unit 31 corrects the boundary portion between the side branch region and the main trunk region with reference to the contour line of the main trunk region determined in S123 (S124). FIG. 11 is a diagram for explaining a boundary portion correction method. By the processing of S121 to S123 described above, the boundary line of the main trunk region is determined. In the example of FIG. 11, the hatched region below the boundary line of the main trunk region is the main trunk region, the dotted region above the boundary line is the side branch region, and the vicinity of the boundary is partially enlarged. At the time of executing the processing of S123, the boundary of the main trunk region has been determined, but the boundary of the side branch region has not been determined, and moreover, whether the main trunk or the side branch is determined on a pixel basis. Therefore, a void may be generated between the boundary line of the main trunk region and the side branch region. In the image segmentation, each pixel is classified into a main trunk or a side branch, and a void represents a region including pixels not classified into the main trunk or the side branch (i.e., pixels with blank labels). Note that, in the image segmentation, in a case where each pixel is classified into a main trunk, a side branch, and a background, the void represents a region including a pixel to which a background label is given or a pixel to which a label is not given. In S124, a process of filling the void in the boundary portion is performed. Specifically, the control unit 31 can fill the void by changing the label of the pixel in the void from “background” or “no label” to “side branch”. After filling the voids, the control unit 31 may temporarily change the main trunk region to a side branch region, and perform processing of subtracting the original main trunk region from the side branch region obtained after the change.

The control unit 31 selects a plurality of points on the peripheral edge from the side branch region whose boundary portion has been corrected (S125). In a case where all the eight pixels adjacent to the pixel of interest among the pixels belonging to the side branch belong to the side branch, the pixel of interest is recognized as a pixel inside the region, and in a case where one or more of the eight pixels adjacent to the pixel of interest belong to the main trunk or the background, the pixel of interest is recognized as a pixel on the peripheral edge. The control unit 31 selects a predetermined number of pixels from among the pixels recognized as pixels on the peripheral edge among the pixels belonging to the side branch region. The number of pixels to be selected may be set in advance or may be set according to the size of the side branch region. The pixel selected in S125 is referred to as a contour point of the side branch region.

The control unit 31 removes contour points in contact with the main trunk region from among the contour points of the side branch region selected in S125 (S126), and determines the contour line of the side branch region based on the remaining contour points (S127). That is, the control unit 31 determines the contour line of the side branch region in the non-boundary portion by obtaining an approximate curve such as a spline curve passing through the contour points (or the vicinity of the contour points) of the non-boundary portion among the contour points of the side branch region.

The control unit 31 determines the contour line of the side branch region at the boundary portion from the contour points included in the boundary portion (S128). That is, the control unit 31 determines the contour line of the side branch region at the boundary portion by obtaining an approximate curve such as a spline curve passing through the contour points (or the vicinity of the contour points) of the boundary portion among the contour points of the side branch region. The control unit 31 determines the contour line of the entire side branch region based on the contour line of the non-boundary portion determined in S127 and the contour line of the boundary portion determined in S128 (S129).

As described above, in the first embodiment, since the contour line of the side branch region is determined with reference to the contour line of the main trunk region, the region (i.e., the boundary line) at the branch portion where the main trunk and the side branch are connected can be defined and presented to the user.

In the present embodiment, the contour lines of the main trunk region and the side branch region are determined and displayed in different display modes, but the control unit 31 may calculate the areas and diameters of the main trunk region and the side branch region and display the calculated values of the areas and diameters. The control unit 31 can count the number of pixels in each region and calculate the area of each region based on the count value. In addition, since the contour line of each region is represented by a mathematical formula such as a spline curve, the control unit 31 can obtain, for example, a linear distance from coordinates indicating the rotation center of the IVUS sensor 12a to the contour line of the main trunk region or the side branch region by computation, and can determine the obtained linear distance as the diameter of each region. Note that the diameter calculated by the control unit 31 may be any of the minimum diameter, the average diameter, and the maximum diameter.

The control unit 31 can determine the contour lines of the main trunk region and the side branch region from the tomographic image of each frame. The control unit 31 may generate a three-dimensional image of the main trunk and side branches based on the contour line determined from each frame. The control unit 31 can display the main trunk and the side branches as wire frames (i.e., three-dimensional images) by displaying the contour lines determined from the respective frames side by side in the long axis direction of the blood vessel. In addition, the control unit 31 may calculate the centroid of the contour line determined from each frame and render the contour line along the trajectory of the calculated centroid to display the main trunk and side branches as a surface model of a three-dimensional image.

Furthermore, since the control unit 31 detects the main trunk region and the side branch region from the tomographic image of each frame, when displaying the frame in which the side branch region is detected on the display apparatus 4, character information of “with side branch”, an icon indicating the presence of the side branch, and the like may be displayed together.

Furthermore, when a side branch is detected in a plurality of consecutive frames and there is a frame in the middle of which a side branch is not detected, the control unit 31 may display character information or an icon indicating that detection omission has occurred on the display apparatus 4.

### Second Embodiment

In the second embodiment, a method for determining a contour line in a case where a side branch comes into contact with a visual field boundary will be described.

Since the overall configuration of the image diagnosis system 100, the internal configuration of the image processing apparatus 3, and the like in the second embodiment are similar to those of the first embodiment, the description thereof will be omitted.

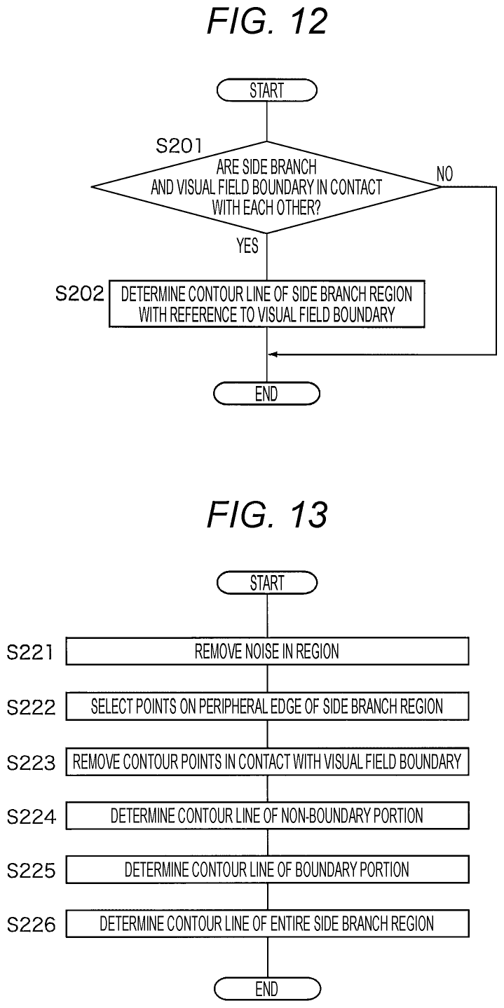

FIG. 12 is a flowchart illustrating a determination procedure of a contour line in a case where a side branch comes into contact with a visual field boundary. The control unit 31 of the image processing apparatus 3 executes the following processing at an appropriate timing after it is determined in S105 of the flowchart illustrated in FIG. 8 that a side branch region is included. For example, the control unit 31 may execute the following processing before the procedure of S107 of determining whether a main trunk region and a side branch region are in contact with each other, or may execute the following processing after the procedure of S107. Furthermore, the control unit 31 may execute the following processing simultaneously in parallel with the procedure of S107.

The control unit 31 determines whether the side branch region is in contact with the visual field boundary (S201). Here, the visual field boundary is a visual field boundary at the time of display, and the display depth (i.e., the maximum diameter displayed from the catheter center) can be changed according to setting by the user. Note that the data of the tomographic image may include data of a depth deeper than the display depth. Furthermore, regarding the depth, the data used for three-dimensional display does not necessarily match the depth of two-dimensional display. From the viewpoint of grasping the entire image of the blood vessel, it is preferable to use data having a deeper depth. When it is determined in S201 that the side branch region is not in contact with the visual field region (S201: NO), the control unit 31 ends the processing according to this flowchart.

When determining that the side branch region is in contact with the visual field region (S201: YES), the control unit 31 determines the contour line of the side branch region with reference to the boundary line of the visual field region (S202). A procedure for determining the contour line of the side branch region with reference to the boundary line of the visual field region will be described in detail with reference to a flowchart illustrated in FIG. 13.

FIG. 13 is a flowchart illustrating a procedure for determining the contour line of the side branch region with reference to the boundary line of the visual field boundary. The control unit 31 refers to the recognition result of S104 and removes noise in the recognized region (S221). A method of removing noise is similar to that of the first embodiment. In a case where S121 of FIG. 10 has already been executed, the control unit 31 may omit the procedure of S221.

The control unit 31 selects a plurality of points on the peripheral edge of the side branch region (S222). In the present embodiment, a set of pixels determined to belong to a side branch is recognized as a side branch region. In a case where all the eight pixels adjacent to the pixel of interest among the pixels belonging to the side branch belong to the side branch, the pixel of interest is recognized as a pixel inside the side branch region, and in a case where one or more of the eight pixels adjacent to the pixel of interest belong to the main trunk or the background, or in a case where the number of pixels adjacent to the pixel of interest is less than eight (in a case where there is no peripheral pixel), the pixel of interest is recognized as a pixel on the peripheral edge. The control unit 31 selects a predetermined number of pixels from among the pixels recognized as pixels on the peripheral edge among the pixels belonging to the side branch region. The number of pixels to be selected may be set in advance or may be set according to the size of the side branch region. The pixel selected in S222 is referred to as a contour point of the side branch region.

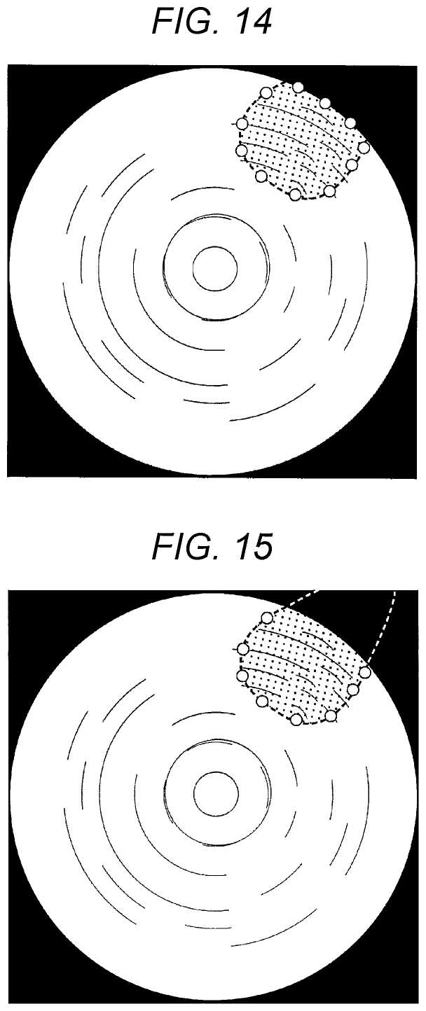

FIG. 14 is a schematic diagram illustrating selection of contour points in a side branch region. In the rectangular region illustrated in FIG. 14, a region surrounded by a large circle is a visual field region, and regions painted in black at the four corners are regions outside the visual field. In the example of FIG. 14, for the sake of simplicity, only the side branch region in contact with the boundary of the visual field region is illustrated. In this example, since the side branch region is in contact with the boundary of the visual field region, not only points on the boundary line between the side branch and the background but also points on the visual field boundary (i.e., points on a large circle) are selected as points on the peripheral edge of the side branch region.

The control unit 31 removes contour points in contact with the visual field boundary from among the plurality of points selected in S222 (S223), and determines the contour line of the side branch region based on the remaining contour points (S224). That is, the control unit 31 determines the contour line of the side branch region in the non-boundary portion by obtaining an approximate curve such as a spline curve passing through the contour points (or the vicinity of the contour points) of the non-boundary portion among the contour points of the side branch region.

The control unit 31 determines the contour line of the side branch region at the boundary portion from the contour points included in the boundary portion (S225). That is, the control unit 31 determines the contour line of the side branch region at the boundary portion by obtaining an approximate curve such as a spline curve passing through the contour points (or the vicinity of the contour points) of the boundary portion among the contour points of the side branch region. The control unit 31 determines the contour line of the entire side branch region based on the contour line of the non-boundary portion determined in S224 and the contour line of the boundary portion determined in S225 (S226).

In the flowchart of FIG. 13, the contour line determination procedure in a case where the side branch region comes into contact with the visual field boundary has been described. However, in a case where the side branch region further comes into contact with the main trunk region, the method described in the first embodiment may be applied, the contour line of the side branch region may be determined with reference to the visual field boundary for the boundary portion with the visual field boundary, and the contour line of the side branch region may be determined with reference to the contour line of the main trunk region for the boundary portion with the main trunk region.

Furthermore, when determining the contour line of the non-boundary portion in S224, the control unit 31 may extrapolate the contour line to a region outside the visual field region, estimate the contour line extending outside the visual field region, and display the estimated contour line outside the visual field region as a virtual line. FIG. 15 is a schematic diagram illustrating an example in which a contour line outside the visual field region is displayed by a virtual line. In this example, a contour line in the visual field region is indicated by a black broken line, and a contour line estimated to exist outside the visual field region is indicated by a white broken line. In this example, since it is estimated that the contour line extends outside the visual field region, it is not necessary to determine the contour line at the boundary portion with the visual field boundary, and the process of S225 may be omitted.

As described above, in the second embodiment, since the contour line of the side branch region is determined with reference to the visual field boundary, the region at the boundary portion where the side branch (i.e., the boundary line) and the visual field boundary are in contact can be defined and presented to the user.

### Third Embodiment

In the third embodiment, an annotation tool used at the time of training the learning model MD will be described.

Since the overall configuration of the image diagnosis system 100, the internal configuration of the image processing apparatus 3, and the like in the third embodiment are similar to those of the first embodiment, the description thereof will be omitted.

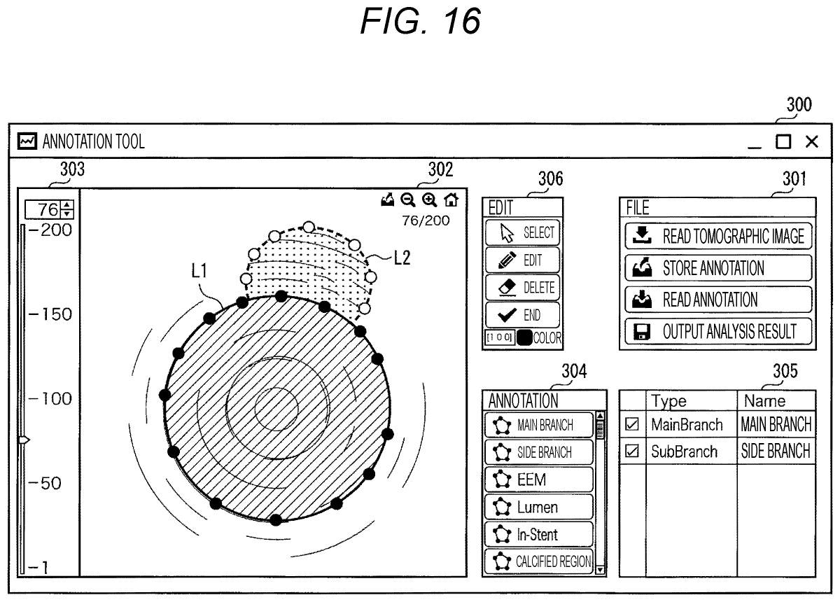

In the present embodiment, the annotation for a large number of tomographic images is performed in the training phase before the recognition processing by the learning model MD is started. Specifically, in the image processing apparatus 3, the annotation tool AT (see FIG. 16) is activated, and the annotation is received in the working environment provided by the tool. The annotation tool AT is one of computer programs installed in the image processing apparatus 3.

FIG. 16 is a diagram for explaining a work environment of annotation.

When the annotation tool AT is activated, the image processing apparatus 3 displays a work screen 300 as illustrated in FIG. 16 on the display apparatus 4. The work screen 300 includes a file selection tool 301, an image display field 302, a frame designation tool 303, a region designation tool 304, a segment display field 305, an editing tool 306, and the like, and receives various operations through the input apparatus 5.

The file selection tool 301 is a tool for receiving selection operations of various files, and includes software buttons for reading a tomographic image, storing annotation data, reading annotation data, and outputting an analysis result. When the tomographic image is read by the file selection tool 301, the read tomographic image is displayed in the image display field 302. The tomographic image generally includes a plurality of frames. The frame designation tool 303 includes an input box and a slider for designating a frame, and is configured to be able to designate a frame of a tomographic image to be displayed in the image display field 302. The example of FIG. 16 illustrates a state in which the 76th frame among 200 frames is designated.

The region designation tool 304 is a tool for receiving designation of a region for the tomographic image displayed in the image display field 302, and includes software buttons corresponding to the respective labels. In the example of FIG. 16, software buttons corresponding to the labels “main trunk” and “side branch” are illustrated. The number of software buttons and the type of label are not limited to the above, and software buttons corresponding to labels such as “EEM”, “Lumen”, “In-Stent”, “calcified region”, “plaque region”, and “thrombus region” may be arranged, and an arbitrary label may be set by the user.

When designating the main trunk region using the region designation tool 304, the user selects the software button labeled “main trunk” and plots a plurality of points so as to surround the main trunk region on the image display field 302. The same applies to the case of designating other regions. The control unit 31 of the image processing apparatus 3 derives a closed curve passing through a plurality of points (or the vicinity of the points) plotted by the user, and draws the derived closed curve in the image display field 302. The interior of the closed curve is drawn in a preset color (or a color set by the user).

The example of FIG. 16 illustrates a state in which the main trunk region is designated by a closed curve L1 passing through a plurality of points indicated by black circles, and the side branch region is designated by a closed curve L2 passing through a plurality of points indicated by white circles. The control unit 31 gives a label “main trunk” to pixels included in the designated main trunk region, and gives a label “side branch” to pixels included in the designated side branch region.

The segment display field 305 displays information of a region drawn in the image display field 302. The example of FIG. 16 illustrates that the main trunk region and the side branch region are displayed in the image display field 302.

The editing tool 306 is a tool for accepting editing of a region drawn in the image display field 302, and includes a selection button, an edit button, an erase button, an end button, and a color setting field. By using the editing tool 306, with respect to the region already drawn in the image display field 302, it is possible to move, add, and erase points that define the region, and change the color of the region.

When saving of the annotation is selected by the file selection tool 301 after the designation or editing of the region is completed, the control unit 31 stores a data set (i.e., annotation data) including the data of the tomographic image and the data of the label attached to each region in the auxiliary storage unit 35.

In the present embodiment, the annotation is performed by manual work of the user. However, if learning of the learning model MD progresses, the annotation can be performed using the recognition result of the learning model MD. In addition, identification information such as presence/absence of frame-out and presence/absence of side branch may be stored in the auxiliary storage unit 35 for learning in units of frames.

Specifically, the control unit 31 displays the acquired tomographic image in the image display field 302, performs region recognition using the learning model MD in the background, calculates a plurality of points passing through the contour of the recognized region, and plots the points in the image display field 302. When the main trunk region and the side branch region are in contact with each other, the control unit 31 may determine the contour line of the side branch region with reference to the contour line of the main trunk region and plot points on each contour line by a procedure similar to that in the first embodiment. In addition, when the side branch region and the visual field boundary are in contact with each other, the control unit 31 may determine a contour line of the side branch region with reference to the visual field boundary and plot points on each contour line by a procedure similar to that of the second embodiment.

Since the image processing apparatus 3 grasps the type of the recognized region, it is possible to automatically assign a label to the region. The image processing apparatus 3 accepts editing of the points plotted in the image display field 302 as necessary, and stores data of a region surrounded by the finally determined points and a label of the region in the auxiliary storage unit 35 as annotation data.

As described above, in the third embodiment, the training data can be easily generated using the annotation tool.

It should be understood that the embodiments disclosed herein are illustrative in all respects and are not restrictive. The scope of the present invention is defined not by the meanings described above but by the claims, and is intended to include meanings equivalent to the claims and all modifications within the scope.

What is claimed is:

1. An image diagnostic system comprising:
a catheter insertable into a blood vessel;
a memory that stores a program;
a display; and
a processor configured to execute the program to:
control the catheter to acquire a tomographic image of a blood vessel,
determine a first region corresponding to a main trunk of the blood vessel and a second region corresponding to a side branch of the blood vessel in the acquired image, and determine whether the first and second regions are connected to each other,
upon determining that the first and second regions are connected to each other, determine a first contour line of the first region in the acquired image,
determine a second contour line of the second region in the acquired image based on the first contour line, and
control the display to display the first and second contour lines on the acquired image using different display modes.

2. The image diagnostic system according to claim 1, wherein
the processor executes the program to input the acquired image into a computer model to generate information indicating the first and second regions, the computer model having been trained with a plurality of tomographic images of blood vessels and a plurality of information each specifying a region corresponding to a main trunk of the blood vessel and a region corresponding to a side branch of the blood vessel in a corresponding one of the tomographic images.

3. The image diagnostic system according to claim 1, wherein
the processor executes the program to, after determining the first contour line and before determining the second contour line:
determine a boundary portion of the second region that is adjacent to the first region, and
correct a shape of the boundary portion so that the boundary portion aligns with the first contour line.

4. The image diagnostic system according to claim 3, wherein
the processor corrects the shape of the boundary portion by merging a region located between the first and second regions into the boundary portion.

5. The image diagnostic system according to claim 3, wherein
the processor executes the program to:
determine a plurality of points on a peripheral edge of the second region, connect one or more of the points located in a portion of the second region other than the boundary portion to form a first line,
connect one or more of the points located in the boundary portion to form a second line, and
combine the first and second lines to form the second contour line.

6. The image diagnostic system according to claim 1, wherein
the acquired image includes a visual field area in which the blood vessel is imaged and a non-visual field area outside the visual field area,
the processor executes the program to determine whether a portion of the second region abuts a boundary between the visual field area and the non-visual field area, and
the second contour line is determined based on the boundary.

7. The image diagnostic system according to claim 6, wherein
the processor executes the program to:
determine a first plurality of points on a peripheral edge of the second region,
remove one or more of the points located along the boundary, and
connect the points from which said one or more of the points have been removed to form a contour line of the second region inside the visual field area.

8. The image diagnostic system according to claim 7, wherein
the processor executes the program to:
determine a virtual contour line of the second region outside the visual field area based on the contour line of the second region inside the visual field area, and
control the display to display the virtual contour line together with the contour line of the second region inside the visual field area.

9. The image diagnostic system according to claim 1, wherein
the processor executes the program to calculate an area or a diameter of the first and second regions using the first and second contour lines.

10. The image diagnostic system according to claim 1, wherein
the processor executes the program to:
generate a three-dimensional image of the blood vessel based on the first and second contour lines, and
control the display to display the three-dimensional image.

11. The image diagnostic system according to claim 1, wherein
the first and second contour lines are displayed using lines of different styles, widths, or colors.

12. The image diagnostic system according to claim 1, wherein
the processor controls the display to display the first and second regions on the acquired image using different hatching patterns.

13. An image diagnostic method comprising:
acquiring a tomographic image of a blood vessel;
determining a first region corresponding to a main trunk of the blood vessel and a second region corresponding to a side branch of the blood vessel in the acquired image, and determining whether the first and second regions are connected to each other;
upon determining that the first and second regions are connected to each other, determining a first contour line of the first region in the acquired image;
determining a second contour line of the second region in the acquired image based on the first contour line; and
displaying the first and second contour lines on the acquired image using different display modes.

14. The image diagnostic method according to claim 13, wherein
said determining the first region and the second region includes inputting the acquired image into a computer model to generate information indicating the first and second regions, the computer model having been trained with a plurality of tomographic images of blood vessels and a plurality of information each specifying a region corresponding to a main trunk of the blood vessel and a region corresponding to a side branch of the blood vessel in a corresponding one of the tomographic images.

15. The image diagnostic method according to claim 13, wherein
said determining the second contour line includes:
determining a boundary portion of the second region that is adjacent to the first region, and
correcting a shape of the boundary portion so that the boundary portion aligns with the first contour line.

16. The image diagnostic method according to claim 15, wherein
said correcting includes merging a region located between the first and second regions into the boundary portion.

17. The image diagnostic method according to claim 15, wherein
said determining the second contour line includes:
determining a plurality of points on a peripheral edge of the second region,
connecting one or more of the points located in a portion of the second region other than the boundary portion to form a first line,
connecting one or more of the points located in the boundary portion to form a second line, and
combining the first and second lines to form the second contour line.

18. The image diagnostic method according to claim 13, wherein
the acquired image includes a visual field area in which the blood vessel is imaged and a non-visual field area outside the visual field area,
said determining the second contour line includes determining whether a portion of the second region abuts a boundary between the visual field area and the non-visual field area, and
the second contour line is determined based on the boundary.

19. The image diagnostic method according to claim 18, wherein
said determining the second contour line includes:
determining a first plurality of points on a peripheral edge of the second region,
removing one or more of the points located along the boundary, and
connecting the points from which said one or more of the points have been removed to form a contour line of the second region inside the visual field area.

20. A non-transitory computer readable storage medium storing a program causing a computer to execute an image diagnostic method comprising:
acquiring a tomographic image of a blood vessel;
determining a first region corresponding to a main trunk of the blood vessel and a second region corresponding to a side branch of the blood vessel in the acquired image, and determining whether the first and second regions are connected to each other;
upon determining that the first and second regions are connected to each other, determining a first contour line of the first region in the acquired image;
determining a second contour line of the second region in the acquired image based on the first contour line; and
displaying the first and second contour lines on the acquired image using different display modes.
